(19) United States
(12) Patent Application Publication
(10) Pub. No.: US 2022/0003454 A1
(43) Pub. Date: Jan. 6, 2022

(54) DOUBLE-LAYER SOUND INSULATION AND NOISE REDUCTION DEVICE AND ATOMIZER

(71) Applicant: SHENZHEN QIANHAI PATUOXUN NETWORK AND TECHNOLOGY CO LTD, Shenzhen (CN)

(72) Inventors: Chengwu Wang, Shenzhen (CN); Tingsheng Wang, Shenzhen (CN)

(21) Appl. No.: 17/355,535

(22) Filed: Jun. 23, 2021

(30) Foreign Application Priority Data
Jul. 1, 2020 (CN) 202010628053.3

(51) Int. Cl. F24F 13/24; F24F 6/12; G10K 11/162

(52) U.S. Cl. CPC F24F 13/24; F24F 2013/242; G10K 11/162; F24F 6/12

(57) ABSTRACT

Provided are a double-layer sound insulation and noise reduction device and an atomizer. The double-layer sound insulation and noise reduction device includes a noise reduction main body, a water baffle and a connecting member, the noise reduction main body includes an annular outer wall and an annular inner wall, the annular inner wall is spaced apart from the annular outer wall to form a sound insulation cavity, and the connecting member is connected between the annular inner wall and the annular outer wall; a side of the annular outer wall is provided with an outer wall ventilation opening, a side of the annular inner wall is provided with an inner wall ventilation opening communicating with the outer wall ventilation opening, and a bottom of the annular inner wall is formed with a water passing hole.

### CROSS-REFERENCE TO RELATED APPLICATION

This application claims priority to Chinese Patent Application No. 202010628053.3 filed Jul. 1, 2020, the disclosure of which is incorporated herein by reference in its entirety.

### TECHNICAL FIELD

The present disclosure belongs to the technical field of household appliances, and in particular relates to a double-layer sound insulation and noise reduction device and an atomizer.

### BACKGROUND

With the improvement of people's living standard, the understanding of the concept of air quality and demands of air quality, atomized products, such as a humidifier or an aromatherapy machine, are gradually accepted by people. The humidifier can increase the humidity in the air to solve a series of problems caused by dry environment, and thus become common household equipment. The aromatherapy machine can also generate an aromatherapy effect in addition to properly increasing the humidity of the air, which is beneficial to sleeping, mosquito repelling, breath smoothing, etc. Most of these products adopt an ultrasonic atomization technology.

The ultrasonic humidifier and aromatherapy machine use ultrasonic electronic high-frequency oscillation to cause the resonance of a ceramic atomization sheet, so that ultrasonic waves are generated. A cavitation phenomenon is generated when the ultrasonic waves are propagated in water. The cavitation phenomenon includes three stages, namely, formation, growth and violent collapse of cavitation bubbles. An atomizer crushes water around the cavitation bubbles into particles of 1 μm to 3 μm by utilizing huge instantaneous pressure generated at the moment of collapse of the cavitation bubbles, so that water mist is generated. When atomization is performed by an ultrasonic atomization sheet, water drops may rush upwards and then fall back to a water tank after reaching a certain height, and due to a fact that the water drops vertically move up and down, in a falling process of the water drops, the water drops collide with a water surface to generate obvious noise. The drip of water is especially obvious in the night, which greatly affects the use experience of a user. Further, if the noise is too high, the noise may affect people's cardiovascular function and hormone secretion function, and even have a great negative effect on people's psychology.

### SUMMARY

The present disclosure aims to provide a double-layer sound insulation and noise reduction device and an atomizer, and is intended to solve a technical problem that an atomizer in the related art generates excessive noise. The present disclosure provides a double-layer sound insulation and noise reduction device. The double-layer sound insulation and noise reduction device includes a noise reduction main body, a water baffle and a connecting member. The noise reduction main body includes an annular outer wall and an annular inner wall located within the annular outer wall, and the annular inner wall is spaced apart from the annular outer wall to form a sound insulation cavity. The connecting member is connected between the annular inner wall and the annular outer wall. A side of the annular outer wall is provided with an outer wall ventilation opening, a side of the annular inner wall is provided with an inner wall ventilation opening communicating with the outer wall ventilation opening. A bottom of the annular inner wall is formed with a water passing hole. The water baffle is disposed to cover a top of the annular inner wall. The side of the annular inner wall is further provided with a mist passing opening close to or extending to the water baffle.

In an embodiment, the connecting member is an inner and outer wall connecting ring, the inner and outer wall connecting ring has an outer ring and an inner ring, a bottom of the annular outer wall is disposed flush with the bottom of the annular inner wall, and the outer ring of the inner and outer wall connecting ring is connected to a bottom edge of the annular outer wall, and the inner ring of the inner and outer wall connecting ring is connected to a bottom edge of the annular inner wall.

In an embodiment, the connecting member is an inner and outer wall connecting beam, the inner and outer wall connecting beam is disposed within the sound insulation cavity, one end of the inner and outer wall connecting beam is connected to an inner surface of the annular outer wall, and the other end of the inner and outer wall connecting beam is connected to an outer surface of the annular inner wall.

In an embodiment, the connecting member is an inner and outer wall connecting platform, the inner and outer wall connecting platform has an outer ring and an inner ring, the top of the annular inner wall extends above the top of the annular outer wall, the outer ring of the inner and outer wall connecting platform is connected to a top edge of the annular outer wall, the inner ring of the inner and outer wall connecting platform is connected to an outer surface of the annular inner wall, and the mist passing opening is located above the inner and outer wall connecting platform.

In an embodiment, the inner and outer wall connecting platform is provided with a water dropping opening communicating with the sound insulation cavity.

In an embodiment, the double-layer sound insulation and noise reduction device further includes a silencing cotton, the silencing cotton is disposed within the sound insulation cavity and is kept away from the mist passing opening, and the silencing cotton is provided with a silencing cotton ventilation opening communicating with the outer wall ventilation opening and the inner wall ventilation opening.

In an embodiment, the connecting member is an inner and outer wall connecting bone, the inner and outer wall connecting bone has an outer ring and an inner ring, a bottom of the annular outer wall is disposed flush with the bottom of the annular inner wall, and the top of the annular inner wall extends above the top of the annular outer wall, the outer ring of the inner and outer wall connecting bone is connected to a bottom edge of the annular outer wall, the inner ring of the inner and outer wall connecting bone is connected to a bottom edge of the annular inner wall, and the mist passing opening is located above a top of the silencing cotton.

In an embodiment, the silencing cotton is of a net-shaped structure.

In an embodiment, the double-layer sound insulation and noise reduction device further includes a honeycomb type silencing chamber, the honeycomb type silencing chamber is disposed within the sound insulation cavity and is kept away from the mist passing opening, the honeycomb type silencing chamber is provided with a silencing chamber ventilation opening and multiple honeycomb holes, and the silencing chamber ventilation opening communicates with the outer wall ventilation opening and the inner wall ventilation opening.

Another embodiment of the present disclosure provides an atomizer. The atomizer includes the double-layer sound insulation and noise reduction device described above.

### BRIEF DESCRIPTION OF DRAWINGS

FIG. 1 is a schematic view of a double-layer sound insulation and noise reduction device in accordance with an embodiment one of the present disclosure.

FIG. 2 is a schematic view of the double-layer sound insulation and noise reduction device of FIG. 1 from another perspective.

FIG. 3 is a sectional view of the double-layer sound insulation and noise reduction device of FIG. 1.

FIG. 4 is a sectional view of an atomizer in accordance with an embodiment six of the present disclosure.

FIG. 5 is a schematic view of a double-layer sound insulation and noise reduction device in accordance with an embodiment two of the present disclosure.

FIG. 6 is a schematic view of the double-layer sound insulation and noise reduction device of FIG. 5 from another perspective.

FIG. 7 is a sectional view of the double-layer sound insulation and noise reduction device of FIG. 5.

FIG. 8 is a schematic view of a double-layer sound insulation and noise reduction device in accordance with an embodiment three of the present disclosure.

FIG. 9 is a schematic view of the double-layer sound insulation and noise reduction device of FIG. 8 from another perspective.

FIG. 10 is a sectional view of the double-layer sound insulation and noise reduction device of FIG. 8.

FIG. 11 is a schematic view of a double-layer sound insulation and noise reduction device in accordance with an embodiment four of the present disclosure.

FIG. 12 is a schematic view of the double-layer sound insulation and noise reduction device of FIG. 11 from another perspective.

FIG. 13 is a sectional view of the double-layer sound insulation and noise reduction device of FIG. 11.

FIG. 14 is an exploded view of the double-layer sound insulation and noise reduction device of FIG. 11.

FIG. 15 is a schematic view of a double-layer sound insulation and noise reduction device in accordance with an embodiment five of the present disclosure.

FIG. 16 is an exploded view of the double-layer sound insulation and noise reduction device of FIG. 15.

Reference list

| | | | |
|---|---|---|---|
| 11 | Ultrasonic atomization sheet | 12 | Water tank |
| 13 | Fan | 14 | Air outlet |
| 15 | Inner cavity air inlet | 16 | Mist outlet |
| 17 | Mist outlet channel | 18 | Sealing ring |
| 19 | Bottom shell | 20 | Upper shell |
| 21 | Circuit board | | |
| 100 | Double-layer sound insulation | | |
| | and noise reduction device | | |
| 101 | Annular outer wall | 102 | Annular inner wall |
| 103 | Water baffle | 104 | Mist passing opening |
| 105 | Outer wall ventilation opening | 106 | Water passing hole |
| 107 | Inner wall ventilation opening | 200 | Noise reduction main |
| | | | body |
| 300 | Sound insulation cavity | 108a | Inner and outer wall |
| | | | connecting ring |
| 108b | Inner and outer wall connecting | | |
| | beam | | |
| 108c | Inner and outer wall connecting | | |
| | platform | | |
| 108d | Inner and outer wall connecting | | |
| | bone | | |
| 109a | silencing cotton | | |
| 109b | honeycomb type silencing | | |
| | chamber | | |
| 1091 | Silencing cotton ventilation | | |
| | opening | | |
| 1092 | Silencing chamber ventilation | | |
| | opening | | |
| 1093 | Honeycomb hole | | |

### DETAILED DESCRIPTION

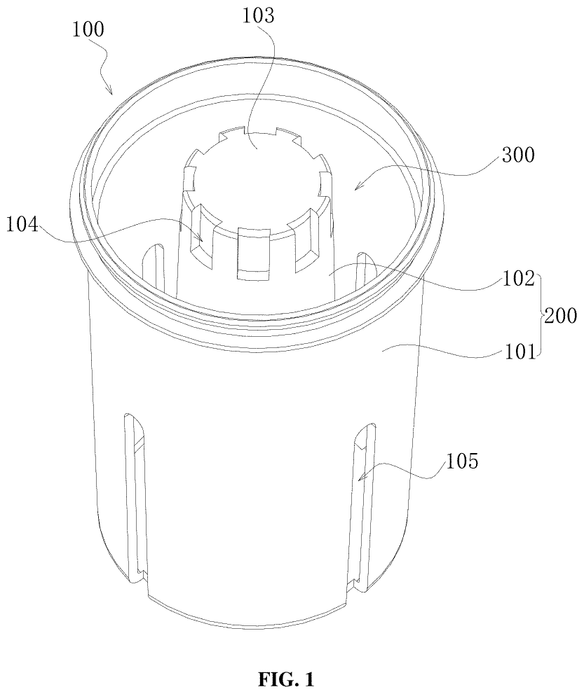

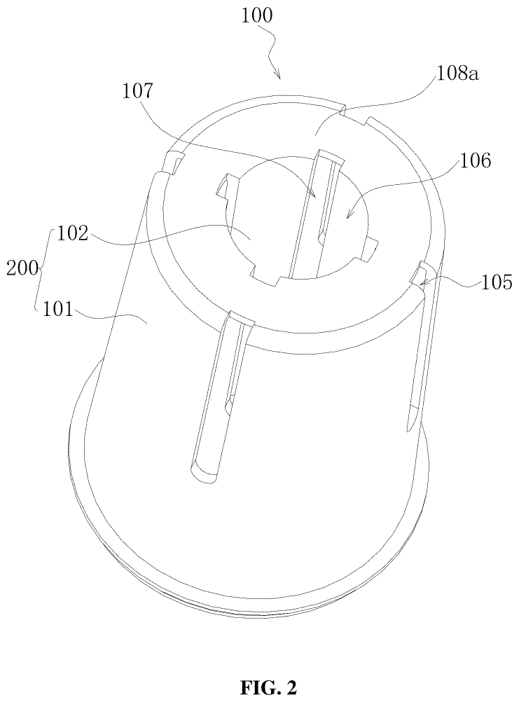

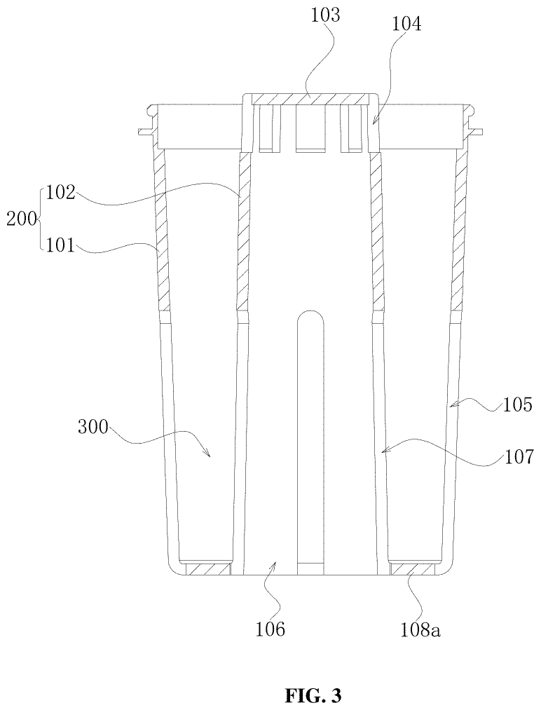

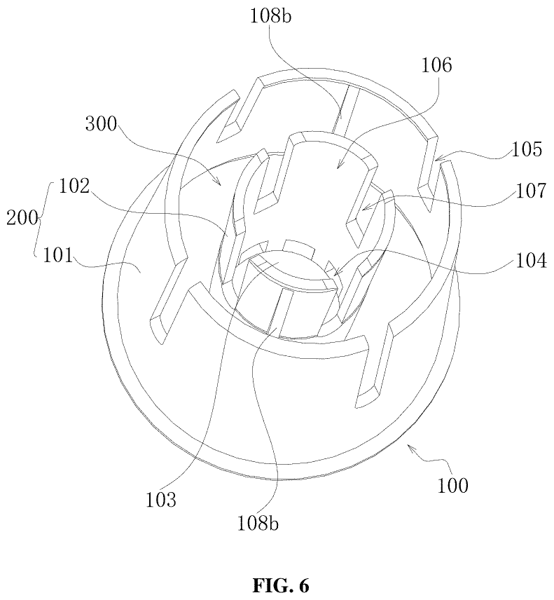

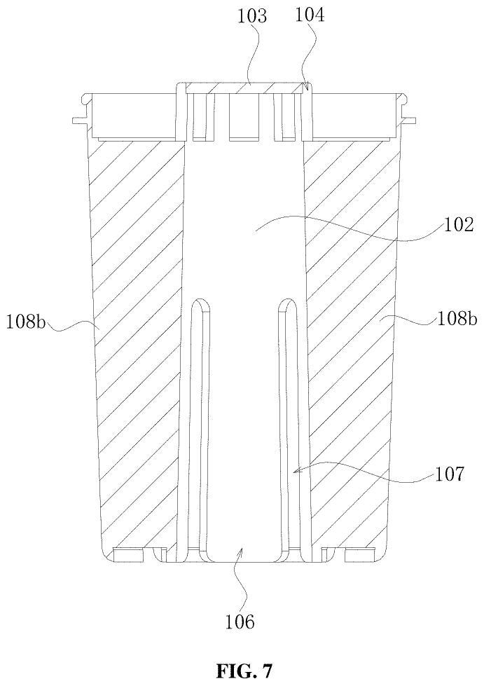

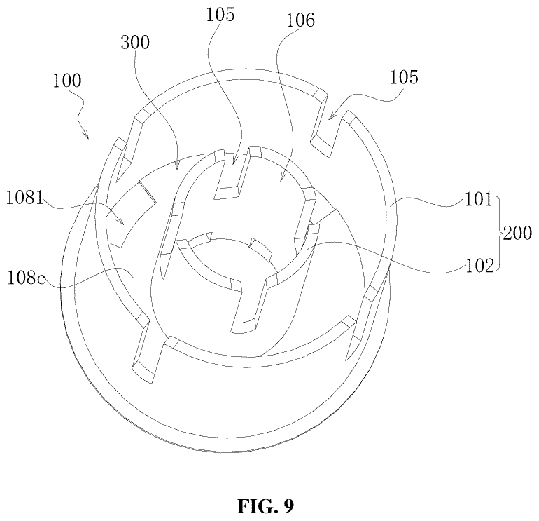

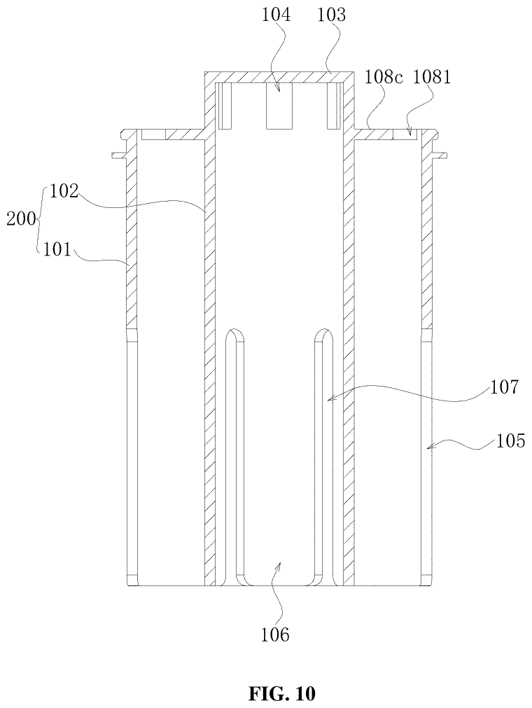

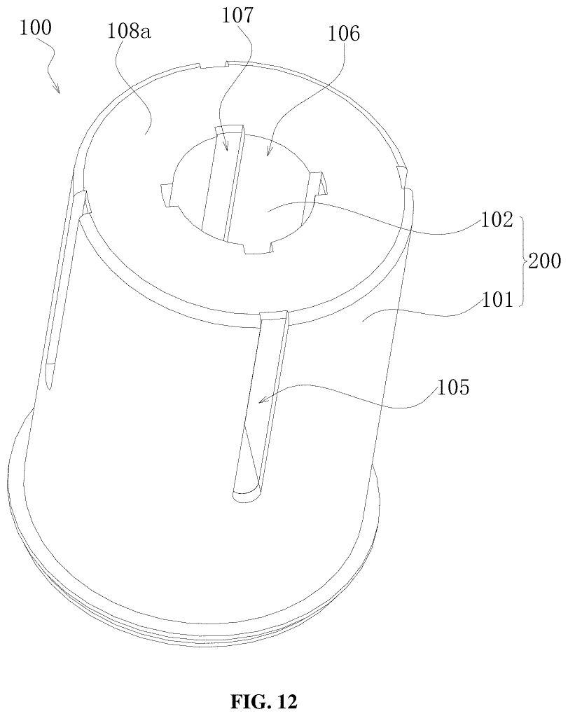

Embodiments of the present disclosure are described in detail below, examples of which are illustrated in the drawings, in which the same or similar reference numerals denote the same or similar elements or elements having the same or similar functions throughout. The embodiments described below with reference to FIGS. 1 to 16 are exemplary and are intended to be used for explaining the embodiments of the present disclosure and are not to be construed as limiting the present disclosure.

In the description of the embodiments of the present disclosure, it is to be understood that orientations or positional relationships indicated by the terms such as “length”, “width”, “upper”, “lower”, “front”, “rear”, “left”, “right”, “vertical”, “horizontal”, “top”, “bottom”, “inner” and “outer” are orientations or positional relationships based on the drawings, and are merely for convenience and simplification of description of the embodiments of the present disclosure, rather than indicating or implying that the referred devices or elements must have a particular orientation, or be constructed and operated in a particular orientation., Thus, these terms should not be construed as limiting the present disclosure.

Moreover, the term “first” and “second” are used for descriptive purposes only and are not to be construed as indicating or implying relative importance or to implicitly indicate the number of indicated technical features. Thus, a feature defined by “first” and “second” may explicitly or implicitly include one or more features. In the description of the embodiments of the present disclosure, the meaning of “multiple” means two or more than two unless expressly specifically defined otherwise.

In the embodiments of the present disclosure, unless explicitly specified or limited otherwise, the terms “mounted”, “interconnected”, “connected”, “fixed” and the like are to be construed broadly, for example may mean fixed connected, detachable connected, or integral; may mean mechanical connected or electrically connected; may mean directly connected, indirectly connected through an intermediate medium, and may mean inside connection of two elements or the interaction between two elements. The specific meaning of the above-described terms in the embodiments of the present disclosure may be understood according to the particular circumstances by those of ordinary skill in the art.

### Embodiment One

In one embodiment of the present disclosure, as illustrated in FIGS. 1 to 3, a double-layer sound insulation and noise reduction device 100 is provided. When the double-layer sound insulation and noise reduction device 100 is applied to an atomizer, water drops may be significantly guided to flow down, thereby achieving a flow guide effect; and meanwhile, due to the double-layer sound insulation, sound wave energy is absorbed inside the atomizer, whereby the sound wave penetration loss amount is significantly increased, the mute effect of the atomizer is achieved, and thus the satisfaction degree of a user for the product experience is improved.

Specifically, the double-layer sound insulation and noise reduction device 100 includes a noise reduction main body 200, a water baffle 103 and a connecting member. The noise reduction main body 200 includes an annular outer wall 101 and an annular inner wall 102 located within the annular outer wall 101. The annular outer wall 101 is a plate member or a housing which is enclosed into a circle and has a ring-shaped cross section. Similarly, the annular inner wall 102 is also a plate member or a housing which is enclosed into a circle and has a ring-shaped cross section, and a cross-sectional area of the annular inner wall 102 is less than a cross-sectional area of the annular outer wall 101, so that the annular inner wall 102 may be disposed within the annular outer wall 101. In this way, a sound insulation cavity 300 is formed between the annular inner wall 102 and the annular outer wall 101 after the annular inner wall 102 is disposed to be spaced apart from the annular outer wall 101.

The annular inner wall 102 and the annular outer wall 101 are connected together through the connecting member, namely, the connecting member is connected between the annular inner wall 102 and the annular outer wall 101. In this way, the entire noise reduction main body 200 forms a unitary, non-loose component.

A distance between the annular inner wall 102 and the annular outer wall 101 is preferably in a range of 5 mm to 10 mm The sound insulation cavity 300 formed between the annular inner wall 102 and the annular outer wall 101 is in a shape of a circular three-dimensional space.

In this embodiment, a connection between the connecting member and the annular inner wall 102 or the annular outer wall 101 may be a detachable connection (such as snap-fitting, buckling and fastener locking connection), or a non-detachable connection (such as fusion, welding and integral forming). In the actual manufacturing process, a suitable connection method may be selected to connect the annular inner wall 102 to the annular outer wall 101 by the connecting member according to production conditions or based on other considerations.

Further, a side of the annular outer wall 101 is provided with an outer wall ventilation opening 105, and a side of the annular inner wall 102 is provided with an inner wall ventilation opening 107 communicating with the outer wall ventilation opening 105. The outer wall ventilation opening 105 and the inner wall ventilation opening 107 may be designed to be a groove shape or a round shape or other shapes. The number of outer wall ventilation openings 105 is more than one, and the number of the inner wall ventilation openings 107 is more than one.

Still further, a bottom of the annular inner wall 102 is formed with a water passing hole 106. In practical application, the water passing hole 106 is located below a water level of a water tank 12 in the atomizer, so that water drops generated in the atomizer flow down to a water surface along the annular inner wall 102, and thus obvious drip of water generated when water directly falls and impacts the water surface is avoided.

Preferably, the water passing hole 106 is closer to a bottom surface of the water tank 12, generally 3 mm to 5 mm Due to a fact that the water drops flow down along the annular inner wall 102 and reach the bottom of the inner wall, namely a position of the water passing opening, a distance between the water passing opening and the water surface is less than 5 mm, a drop impulse of the water drops is small, and human ears cannot hear the drip of water, thereby obviously improving the user experience. However, the water baffle 103 is disposed to cover a top of the annular inner wall 102, a position height of the water baffle 103 is set according to an actual situation of a whole machine product of an atomizer to which the water baffle 103 is applied, and a position of the water baffle 103 of the atomizer with high power may be higher.

The side of the annular inner wall 102 is further provided with a mist passing opening 104 close to or extending to the water baffle 103. That is to say, the mist passing opening 104 disposed at the side of the annular inner wall 102 may directly extend to a bottom surface of the water baffle 103 or extend to a position away from the bottom surface of the water baffle 103 by a small distance.

In this embodiment, the water baffle 103 and the noise reduction main body 200 are preferably of a same component, that is, are integrally formed, so that the overall structure is simpler, and thus the production cost is significantly reduced.

Of course, in other embodiments, the water baffle 103 and the noise reduction main body 200 may be detachably connected.

The double-layer sound insulation and noise reduction device 100 provided by the embodiments of the present disclosure is suitable for use in the atomizer, when the atomizer is working, a ultrasonic atomization sheet 11 of the atomizer vibrates at a high frequency to cause water to vibrate upward and thus pass through the water passing hole 106, and after reaching a certain height, formed water drops are blasted into particles and mist. A fan 13 of the atomizer conveys power air through the outer wall ventilation opening 105 and the inner wall ventilation opening 107 to blow out the mist from the mist passing opening 104 above. Since the sound insulation cavity 300 is formed between the annular inner wall 102 and the annular outer wall 101 of the noise reduction main body 200, when the water drops flow down along an inner surface of the annular inner wall 102, the drip of water appears on the water surface, and sound waves of the water drops pass through the annular inner wall 102, are scattered within the sound insulation cavity 300, and spread in all directions. Therefore, the sound wave energy is obviously consumed inside and around the sound insulation cavity, so that the sound wave penetration loss amount is significantly increased, and a good mute effect is achieved.

The penetration loss amount refers to a difference between incident sound energy and emergent sound energy, and is an important parameter for reflecting a sound insulation effect. The larger the sound wave penetration loss amount is, the better the sound insulation effect is. In this way, the drip of water generated by the atomizer with this double-layer sound insulation and noise reduction device 100 is significantly isolated and consumed, and then the noise transmitted to and heard by the human ear is very small.

In this embodiment, as illustrated in FIGS. 1 to 3, the connecting member is an inner and outer wall connecting ring 108a. The inner and outer wall connecting ring 108a has an outer ring and an inner ring. A shape of the inner and outer wall connecting ring 108a is adapted to a shape of a cross-section of the sound insulation cavity 300. A bottom of the annular outer wall 101 is disposed flush with the bottom of the annular inner wall 102. The outer ring of the inner and outer wall connecting ring 108a is connected to a bottom edge of the annular outer wall 101, and the inner ring of the inner and outer wall connecting ring 108a is connected to a bottom edge of the annular inner wall 102. In this way, the annular inner wall 102 and the annular outer wall 101 may be connected through the inner and outer wall connecting ring 108a, and the water passing hole 106 formed in the bottom of the annular inner wall 102 may not be blocked or affected.

The application of the double-layer sound insulation and noise reduction device 100 provided by this embodiment to the atomizer will be further described as an example.

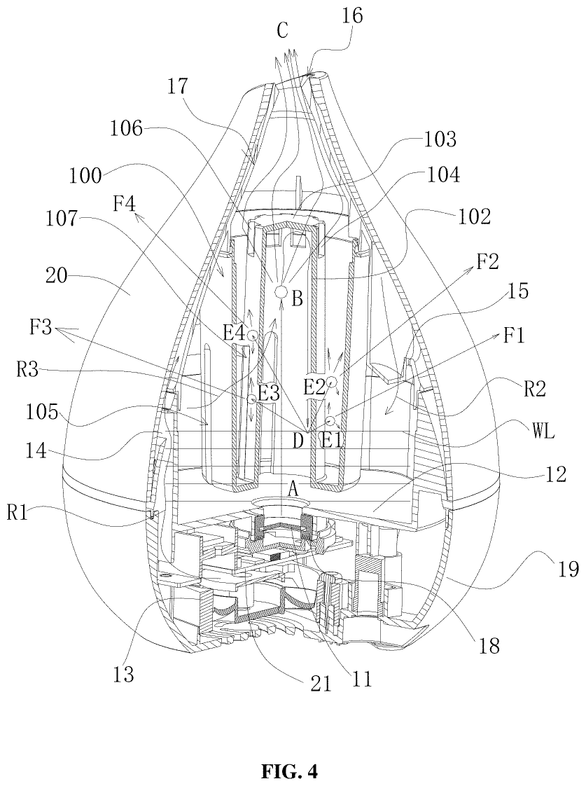

As illustrated in FIG. 4, there is provided an atomizer. The atomizer externally includes a bottom shell 19 and an upper shell 20 which are buckled, and an air outlet 14, an inner cavity air inlet 15, a mist outlet 16 and a mist outlet channel 17 are formed in the bottom shell 19 and the upper shell 20. Moreover, the atomizer is internally provided with the double-layer sound insulation and noise reduction device 100 in the above embodiments, the ultrasonic atomization sheet 11, the water tank 12, the fan 13 and a circuit board 21. When the atomizer works, the ultrasonic atomization sheet 11 vibrates at a high frequency to allow water to vibrate upward from a point A and then pass through the water passing hole 106 to reach a point B below the water baffle 103, and formed water drops are blasted into particles and mist. A sealing ring 18 is disposed on a periphery of the ultrasonic atomization sheet 11.

As illustrated by arrow direction routes of R1, R2 and R3, the fan 13 works to blow out the power air from the air outlet 14, the power air reaches an inner cavity air inlet 15 along an air duct, and the air blows small atomized particles out of the mist passing opening 104 through the outer wall ventilation opening 105 and the inner wall ventilation opening 107. Under an action of air force, the mist keeps going upwards to pass through the mist outlet channel 17 and reaches a position above a point C outside the mist outlet 16, thereby forming a mist spray form in which the mist is scattered upwards is formed.

Further, in the double-layer sound insulation and noise reduction device 100, after the vibrated water drops are blasted at the point B, the water drops are blasted into particles and mist. As described above, the mist is blown away from the point C, while the large particles are dispersed into the water baffle 103 and flow down along the annular inner wall 102 to a point D in a horizontal plane WL to produce the drip of water, where the point D is a source of noise generation where the drip of water occurs. The noise diffuses and propagates at the point D, and part of the noise penetrates into water downwards so that the energy is absorbed. Most of the noise propagates sideways and upwards through the annular inner wall 102 to an interlayer cavity, for example to points E1, E2, E3, E4 illustrated in FIG. 4, the sound waves diverge within the interlayer cavity, and the energy is dispersed and consumed by means of the back and forth impact in walls within the interlayer cavity. The remaining small part of sound wave energy that is unconsumed is propagated out from the points E1, E2, E3, E4 in directions F1, F2, F3, F4, respectively. Since the sound waves are greatly consumed within the interlayer cavity, there is very little residual sound wave energy passing through the annular outer wall 101, so that the user does not hear the significant noise. As can be seen from this, the double-layer sound insulation and noise reduction device 100 provided by this embodiment can significantly increase the sound wave penetration loss amount. The penetration loss amount is the difference between the incident sound energy and the emergent sound energy, and is an important parameter for reflecting the sound insulation effect. The larger the sound wave penetration loss amount is, the better the sound insulation effect is.

### Embodiment Two

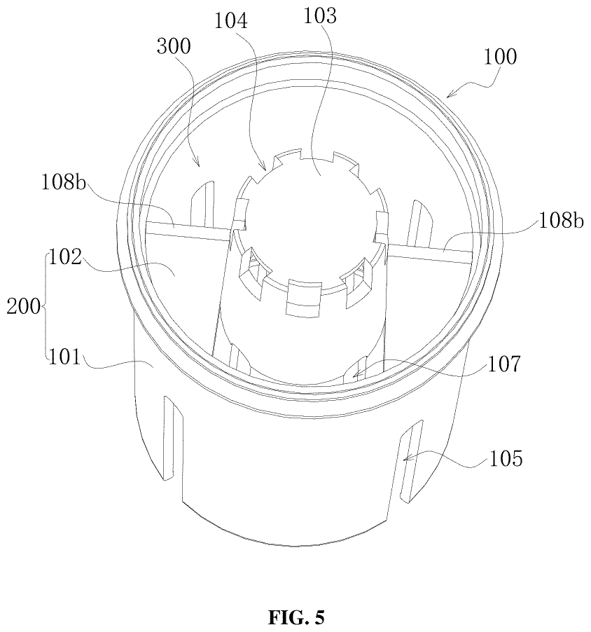

As illustrated in FIGS. 5 to 7, a difference between this embodiment and the embodiment one described above is hat: the connecting member is an inner and outer wall connecting beam 108b, the inner and outer wall connecting beam 108b is disposed within the sound insulation cavity 300, one end of the inner and outer wall connecting beam 108b is connected to an inner surface of the annular outer wall 101, and the other end of the inner and outer wall connecting beam 108b is connected to an outer surface of the annular inner wall 102. In this embodiment, the inner and outer wall connecting beam 108b may include one or more inner and outer wall connecting beams, and the inner and outer wall connecting beam 108b is of a plate member structure with two ends for connection with the annular outer wall 101 and the annular inner wall 102, respectively, which can also ensure that the annular outer wall 101 and the annular inner wall 102 are connected together as a unitary piece.

Further, the inner and outer wall connecting beam 108b may be integrally formed with the annular outer wall 101 and the annular inner wall 102.

The rest of this embodiment is the same as that of the embodiment one, and features that are not explained in this embodiment are all explained in the embodiment one, which will not described herein again.

### Embodiment Three

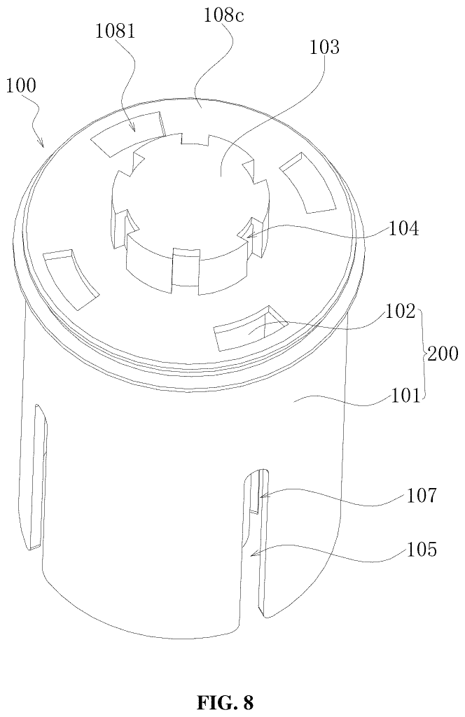

As illustrated in FIGS. 8 to 10, a difference between this embodiment and the embodiment one described above is that: the connecting member is an inner and outer wall connecting platform 108c, the inner and outer wall connecting platform 108c has an outer ring and an inner ring, the top of the annular inner wall 102 extends above the top of the annular outer wall 101, the outer ring of the inner and outer wall connecting platform 108c is connected to a top edge of the annular outer wall 101, the inner ring of the inner and outer wall connecting platform 108c is connected to an outer surface of the annular inner wall 102, and the mist passing opening 104 is located above the inner and outer wall connecting platform 108c. The inner and outer wall connecting platform 108c in this embodiment is similar or identical in structure to the inner and outer wall connecting ring 108a (see FIGS. 1 to 3) in the embodiment one described above, however the inner and outer wall connecting platform 108c is disposed in an upper position of the noise reduction main body 200, which is different from a setting position of the inner and outer wall connecting ring 108a (see FIGS. 1 to 3) in the embodiment one described above. According to this embodiment, the top of the annular inner wall 102 extends to the position above the top of the annular inner wall 102, so that the mist passing opening 104 can be located above the inner and outer wall connecting platform 108c, and when the inner and outer wall connecting platform 108c is connected to an outer annular wall of the annular inner wall 102, the normal going out of the mist from the mist passing opening 104 is not affected, the structural design is ingenious, and the practicability is strong.

As illustrated in FIG. 8, in this embodiment, further, the inner and outer wall connecting platform 108c is provided with a water dropping opening communicating with the sound insulation cavity 300. Specifically, the water dropping opening may enable particle water drops coming out from the mist passing opening 104 to flow back into a double-layer sound insulation cover and flow to the water tank 12 of the atomizer along an inner side of the annular outer wall 101, and therefore recovery of part of the water drops is achieved.

The rest of this embodiment is the same as that of the embodiment one, and features that are not explained in this embodiment are all explained in the embodiment one, which will not described herein again.

### Embodiment Four

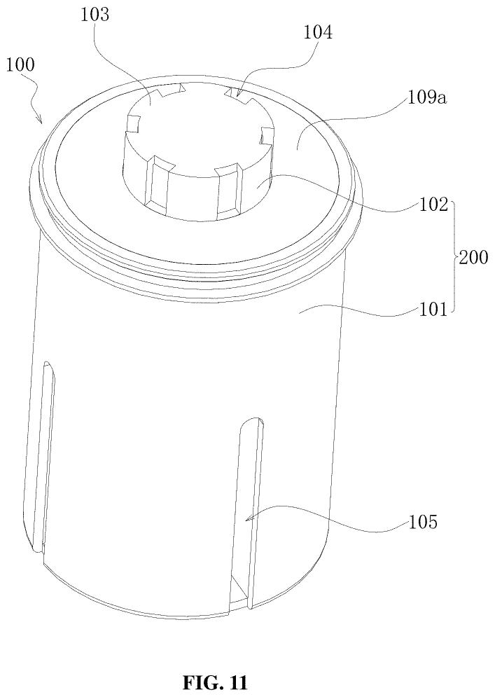

As illustrated in FIGS. 11 to 14, a difference between this embodiment and the embodiment one described above is that: the double-layer sound insulation and noise reduction device 100 further includes a silencing cotton 109a, the silencing cotton 109a is disposed within the sound insulation cavity 300 and is kept away from the mist passing opening 104, so that the disposed silencing cotton 109a does not affect (i.e., does not block) the normal function of the mist passing opening 104. The silencing cotton 109a is provided with a silencing cotton ventilation opening 1091 communicating with the outer wall ventilation opening 105 and the inner wall ventilation opening 107. Specifically, the silencing cotton 109a is used for significantly absorbing sound wave energy propagated into the sound insulation cavity 300, and thus the noise reduction function is achieved. The silencing cotton 109a is provided so that the sound wave energy is further absorbed, noise is further reduced, and the noise reduction performance of the double-layer noise reduction device of the present disclosure is improved.

The silencing cotton 109a may be a sponge, a plant fiber, an artificial inorganic fiber, or the like.

Furthermore, the silencing cotton 109a is of a net-shaped structure.

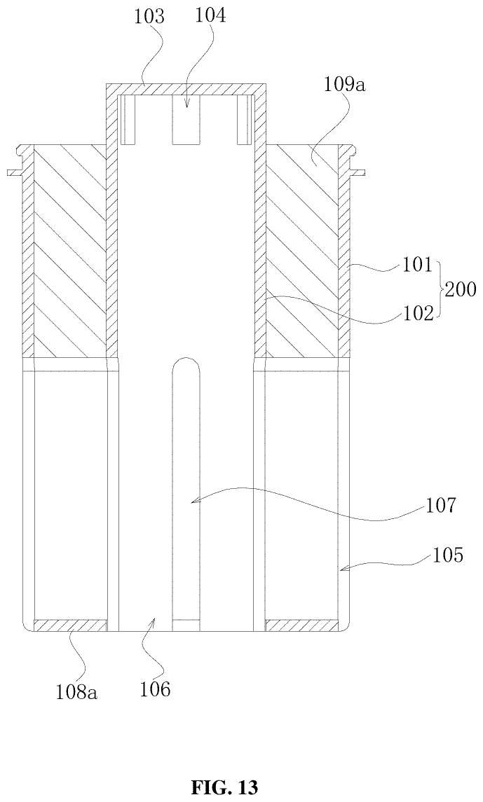

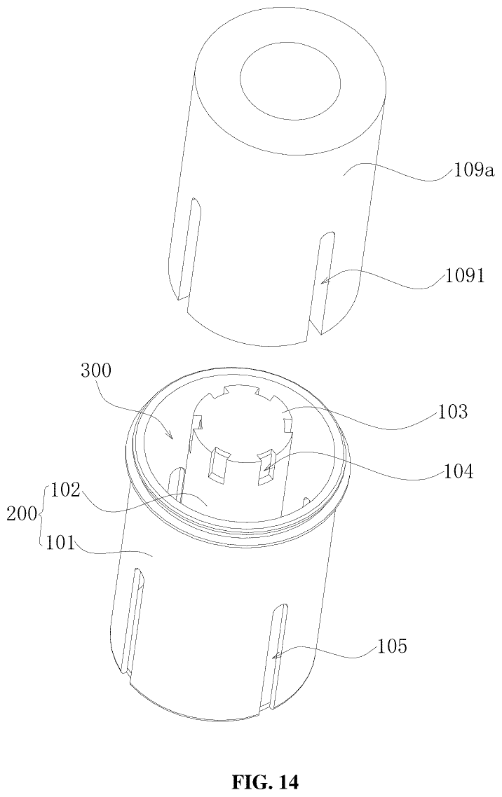

As illustrated in FIGS. 13 to 14, in this embodiment, the connecting member is an inner and outer wall connecting bone 108d, the inner and outer wall connecting bone 108d is similar or identical in structure to the inner and outer wall connecting ring 108a in the embodiment one described above (see FIGS. 1 to 3), and is similar or identical in setting position to the inner and outer wall connecting ring 108a in the embodiment one described above (see FIGS. 1 to 3). Likewise, the inner and outer wall connecting bone 108d in this embodiment has an outer ring and an inner ring, the bottom of the annular outer wall 101 is disposed flush with the bottom of the annular inner wall 102, and the top of the annular inner wall 102 extends above the top of the annular outer wall 101, the outer ring of the inner and outer wall connecting bone 108d is connected to the bottom edge of the annular outer wall 101, the inner ring of the inner and outer wall connecting bone 108d is connected to the bottom edge of the annular inner wall 102, and the mist passing opening 104 is located above a top of the silencing cotton 109a. With such design, it can be guaranteed that the silencing cotton 109a mounted within the sound insulation cavity 300 does not block the mist passing opening 104 disposed close to the top of the annular inner wall 102, the structural design is reasonable, and the practicability is strong.

The rest of this embodiment is the same as that of the embodiment one, and features that are not explained in this embodiment are all explained in the embodiment one, which will not described herein again.

### Embodiment Five

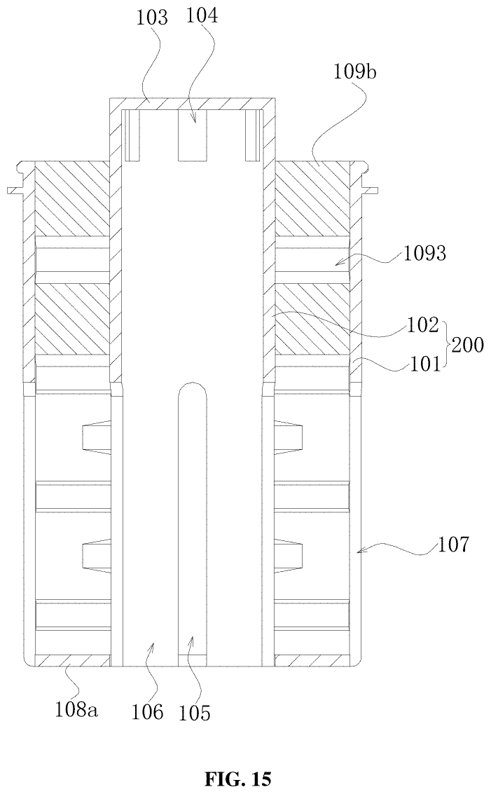

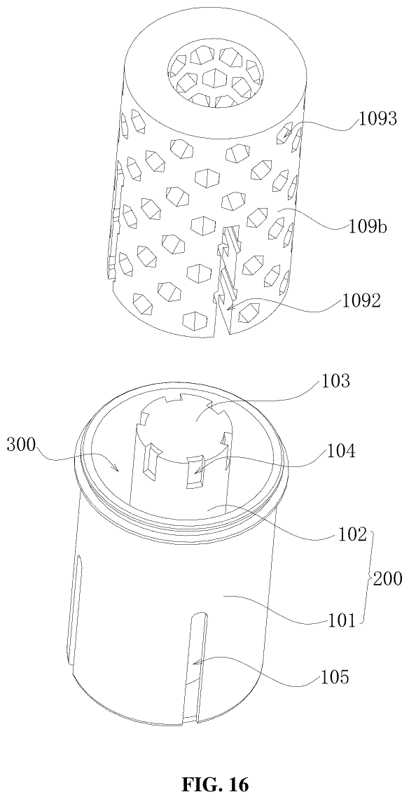

As illustrated in FIGS. 15 to 16, a difference between this embodiment and the embodiment four described above is that: the double-layer sound insulation and noise reduction device 100 further includes a honeycomb type silencing chamber 109b, the honeycomb type silencing chamber 109b is disposed within the sound insulation cavity 300 and is kept away from the mist passing opening 104, the honeycomb type silencing chamber 109b is provided with a silencing chamber ventilation opening 1092 and multiple honeycomb holes 1093, and the silencing chamber ventilation opening 1092 communicates with the outer wall ventilation opening 105 and the inner wall ventilation opening 107. In this embodiment, the honeycomb type silencing chamber 109b is disposed to replace the silencing cotton 109a (as illustrated in FIG. 11) in the embodiment four, the honeycomb holes 1093 of the honeycomb type silencing chamber 109b are regularly arranged, and each of the honeycomb holes 1093 is preferably regular hexagon in shape and may also be a round shape, a square shape or another shape. Due to the design of the honeycomb holes 1093, the sound wave penetration loss amount is significantly increased, and the sound insulation effect is good.

The rest of this embodiment is the same as that of the embodiment one, and features that are not explained in this embodiment are all explained in the embodiment one, which will not described herein again.

### Embodiment Six

As illustrated in FIG. 4, another embodiment of the present disclosure provides an atomizer including the double-layer sound insulation and noise reduction device 100 described above. The atomizer provided by the embodiment of the present disclosure uses the double-layer sound insulation and noise reduction device 100 of any one of the embodiments described above, and with such double-layer sound insulation and noise reduction device 100, the drip of water generated by the atomizer can be significantly isolated and consumed, so that the noise transmitted to and heard by the human ear is very small.

Further, the atomizer provided by the embodiment of the present disclosure may be a humidifier, an aromatherapy machine, or the like.

The foregoing has been presented merely as preferred embodiments of the present disclosure and is not intended to be limiting of the present disclosure, and any modifications, equivalents, and improvements made within the spirit and principles of the present disclosure are intended to be encompassed within the scope of the present disclosure.

What is claimed is:

1. A double-layer sound insulation and noise reduction device, comprising a noise reduction main body, a water baffle and a connecting member, wherein the noise reduction main body comprises an annular outer wall and an annular inner wall located within the annular outer wall, the annular inner wall is spaced apart from the annular outer wall to form a sound insulation cavity, and the connecting member is connected between the annular inner wall and the annular outer wall;
a side of the annular outer wall is provided with an outer wall ventilation opening, a side of the annular inner wall is provided with an inner wall ventilation opening communicating with the outer wall ventilation opening, a bottom of the annular inner wall is formed with a water passing hole, the water baffle is disposed to cover a top of the annular inner wall, and the side of the annular inner wall is further provided with a mist passing opening close to or extending to the water baffle.

2. The double-layer sound insulation and noise reduction device of claim 1, wherein the connecting member is an inner and outer wall connecting ring, the inner and outer wall connecting ring has an outer ring and an inner ring, a bottom of the annular outer wall is disposed flush with the bottom of the annular inner wall, and the outer ring of the inner and outer wall connecting ring is connected to a bottom edge of the annular outer wall, and the inner ring of the inner and outer wall connecting ring is connected to a bottom edge of the annular inner wall.

3. The double-layer sound insulation and noise reduction device of claim 1, wherein the connecting member is an inner and outer wall connecting beam, the inner and outer wall connecting beam is disposed within the sound insulation cavity, one end of the inner and outer wall connecting beam is connected to an inner surface of the annular outer wall, and the other end of the inner and outer wall connecting beam is connected to an outer surface of the annular inner wall.

4. The double-layer sound insulation and noise reduction device of claim 1, wherein the connecting member is an inner and outer wall connecting platform, the inner and outer wall connecting platform has an outer ring and an inner ring, the top of the annular inner wall extends above a top of the annular outer wall, the outer ring of the inner and outer wall connecting platform is connected to a top edge of the annular outer wall, the inner ring of the inner and outer wall connecting platform is connected to an outer surface of the annular inner wall, and the mist passing opening is located above the inner and outer wall connecting platform.

5. The double-layer sound insulation and noise reduction device of claim 4, wherein the inner and outer wall connecting platform is provided with a water dropping opening communicating with the sound insulation cavity.

6. The double-layer sound insulation and noise reduction device of claim 1, further comprising a silencing cotton, wherein the silencing cotton is disposed within the sound insulation cavity and is kept away from the mist passing opening, and the silencing cotton is provided with a silencing cotton ventilation opening communicating with the outer wall ventilation opening and the inner wall ventilation opening.

7. The double-layer sound insulation and noise reduction device of claim 6, wherein the connecting member is an inner and outer wall connecting bone, the inner and outer wall connecting bone has an outer ring and an inner ring, a bottom of the annular outer wall is disposed flush with the bottom of the annular inner wall, the top of the annular inner wall extends above a top of the annular outer wall, the outer ring of the inner and outer wall connecting bone is connected to a bottom edge of the annular outer wall, the inner ring of the inner and outer wall connecting bone is connected to a bottom edge of the annular inner wall, and the mist passing opening is located above a top of the silencing cotton.

8. The double-layer sound insulation and noise reduction device of claim 6, wherein the silencing cotton is of a net-shaped structure.

9. The double-layer sound insulation and noise reduction device of claim 1, further comprising a honeycomb type silencing chamber, wherein the honeycomb type silencing chamber is disposed within the sound insulation cavity and is kept away from the mist passing opening, the honeycomb type silencing chamber is provided with a silencing chamber ventilation opening and a plurality of honeycomb holes, and the silencing chamber ventilation opening communicates with the outer wall ventilation opening and the inner wall ventilation opening.

10. An atomizer, comprising the double-layer sound insulation and noise reduction device of claim 1.

11. An atomizer, comprising the double-layer sound insulation and noise reduction device of claim 2.

12. An atomizer, comprising the double-layer sound insulation and noise reduction device of claim 3.

13. An atomizer, comprising the double-layer sound insulation and noise reduction device of claim 4.

14. An atomizer, comprising the double-layer sound insulation and noise reduction device of claim 5.

15. An atomizer, comprising the double-layer sound insulation and noise reduction device of claim 6.

16. An atomizer, comprising the double-layer sound insulation and noise reduction device of claim 7.

17. An atomizer, comprising the double-layer sound insulation and noise reduction device of claim 8.

18. An atomizer, comprising the double-layer sound insulation and noise reduction device of claim 9.
